(19) United States
(12) Patent Application Publication
(10) Pub. No.: US 2011/0319760 A1
(43) Pub. Date: Dec. 29, 2011

(54) MONITORING SYSTEM

(76) Inventor: Marino Cerofolini, Subbiano (AR) (IT)

(21) Appl. No.: 13/166,863

(22) Filed: Jun. 23, 2011

(30) Foreign Application Priority Data
Jun. 28, 2010 (IT) GE2010A000069

(51) Int. Cl. A61B 8/14

(52) U.S. Cl. 600/443

(57) ABSTRACT

A portable ultrasound monitoring system having an array of electroacoustic transducers, each transducer is constructed and arranged to emit an ultrasound wave when powered with an electric excitation signal and generate an electric reception signal when impinged by a reflected ultrasound wave. The monitoring system also has a generating means for generating the electric excitation signals and a receiving means for receiving the electric reception signals. A supporting means for supporting the portable ultrasound monitoring system is also provided. The supporting means has a fastening means for temporarily fastening at least the array of electroacoustic transducers to a body under examination in a predetermined position for performing an examination.

### CROSS-REFERENCE TO RELATED APPLICATION

This application claims the foreign priority benefit of Italian Patent Application No. GE2010A000069, filed Jun. 28, 2010, which is hereby incorporated by reference.

### BACKGROUND

The present invention generally relates to a portable ultrasound monitoring system.

Currently several types of portable ultrasound systems are known. Such systems have different shape characteristics corresponding to different portability and handiness levels. As known by those in the art, portable computer systems exist which have a sizes ranging from notebook computers down to tablet computers and down to hand-held devices.

However all these systems are always designed for being extemporaneously used during an examination session and when the system is controlled and managed by a third person performing the examination on a patient.

In particular in the cardiology field, and in other fields as well, the patient needs to be subjected to a continuous examination for a certain period of time and during the normal daily life, in order to find pathologies having their effects under specific physical and/or psychological stress conditions. The so called Holter devices are currently known and these are devices that are carried for continuosly performing electrocardiograms. Sensors are applied to the patient which send signals to a continuous recording unit composed of a portable tape.

The developments concerning the electronic field and above all the miniaturization of electronic components and the reduction of their cost, together with the passage from analog to digital mode have allowed recording units to be made more and more small, lightweight and powerful, as well as power consumptions thereof to be reduced prolonging their life. Moreover functions for processing data detected by sensors have been added within said units.

The use of ultrasound imaging diagnostics has been considerably developed also in the cardiology field. The cardiographic imaging allows functional investigations to be made whose information are integrated with those obtained from the ECG leading to better and more accurate diagnostic overviews.

Therefore, by means of relatively simple and inexpensive arrangements, there is the need of providing a monitoring system, in particular for diagnostic purposes, which is very easy to handle and which is able to fit different anatomical parts to be examined, and that, at the same time, is portable such to provide freedom of movement for the patient and which is able to easily interface both with other devices and with remote units for displaying and further processing detected data such that the fact that it is possible to perform examinations continuous in time and without the operator can be extended also in the ultrasound field, allowing the patient to live his/her normal daily life with an inconvenience as little as possible due to the provision of devices necessary for performing the examination.

### SUMMARY OF THE INVENTION

The invention achieves the above aims by providing a monitoring system of the type described hereinbefore providing supporting means in combination with the portable ultrasound system which are composed of further means for temporary fastening to the body such that at least the array of transducers is fastened in the specific position for performing the examination and therefore such that it is integral with the body, or the part thereof, to be examined.

This allows a monitoring system to be provided for performing examinations by using portable systems such to provide the minimum bulkiness and the maximum comfort for the user, while maintaining unchanged the quality of data obtained from the performed examination, moreover having several possibilities for storing and displaying collected data.

In one embodiment of the present disclosure, a portable ultrasound monitoring system having an array of electroacoustic transducers, each transducer is constructed and arranged to emit an ultrasound wave when powered with an electric excitation signal and generate an electric reception signal when impinged by a reflected ultrasound wave. The monitoring system also has a generating means for generating the electric excitation signals and a receiving means for receiving the electric reception signals. A supporting means for supporting the portable ultrasound monitoring system is also provided. The supporting means has a fastening means for temporarily fastening at least the array of electroacoustic transducers to a body under examination in a predetermined position for performing an examination.

Further forms, objects, features, aspects, benefits, advantages, and embodiments of the present invention will become apparent from a detailed description and drawings provided herewith.

### BRIEF DESCRIPTION OF THE DRAWING

FIG. 1 is a schematic diagram of one embodiment of the monitoring system of the present invention.

FIG. 2 is a perspective view of means for temporary fastening to the body which belong to the monitoring system of the present invention according to a possible variant embodiment.

FIGS. 3a and 3b show two possible configurations of the fastening means of the monitoring system of one embodiment of the present invention.

FIGS. 4a and 4b show a possible variant embodiment of the monitoring system of the present invention.

FIGS. 5a and 5b are a perspective view and a side elevation view respectively of a possible configuration of the portable ultrasound system of the monitoring system of the present invention.

FIG. 6 schematically shows one embodiment of monitoring system of the present invention where the portable ultrasound system is provided in combination with an electrocardiograph.

FIGS. 7 and 8 show a variant embodiment of the ultrasound probe according to FIGS. 4 and 5, which variant provides the probe to be divided into two parts that can be removably attached to each other.

### DETAILED DESCRIPTION OF THE PREFERRED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

Except as specifically defined herein, the words/terms used in the claims is to only have its plain and ordinary meaning. Such plain and ordinary meaning is inclusive of all consistent dictionary definitions from the most recently published Webster's dictionaries and Random House dictionaries.

In order to improve the portabilty of the entire ultrasound system, an embodiment of the present invention provides the means for temporary fastening to the body not only to fasten the array of transducers in a specific position, but also to position means for generating the electric excitation signals, means for receiving the electric reception signals, the processing and/or storage unit for the electric excitation and/or reception signals, communication means and power supply means, namely all the constituent elements of the portable ultrasound system, which are connected to the array of transducers by means of communication lines. Such communication lines are also provided integrated within the fastening elements and can transmit data among the several elements of the portable ultrasound system by using cables, or in wireless mode, for example using radio wave transmission.

Advantageously the temporary fastening means on the inside thereof at the portion in contact with the body to be examined have housing spaces, such as for example pockets, intended for housing and keeping in place one or more costituent elements of the portable ultrasound system: such pockets may be secured in specific positions or may be movable such that the portable ultrasound system can meet the different requirements related to the type and condition of the examination to be performed.

Moreover within fastening means there can be provided interfaces between the portable ultrasound system and one or more remote units. This is particularly advantageous, for example, in case the remote unit is an external monitor: the fact that it is possible to establish a communication for transmitting data between the ultrasound system and the display unit, allows the system to be less cumbersome allowing physical properties and/or parameters to be monitored without the space requirements of a display unit. As with communication lines, also the interfaces that are provided can be of the cable type or wireless type, in addition it is possible to provide data to be saved on a storage device, which, in the perspective of reducing the overall dimensions, can be a memory card of the Secure Digital type or the like, which records data from the monitoring system, stores them and allow them to the displayed once it is inserted within the remote display unit.

A preferred embodiment provides the portable ultrasound system to be composed of an ultrasound probe and of control electronics, which are both housed inside the housing pockets of the means for temporary fastening to the body.

There are several possible embodiments that use an ultrasound system as described above and are all the subject of the present invention.

In particular only the array of transducers can be provided within the probe, that by means of communication lines receives and sends signals from and to the control electronics and consequently means for generating electric excitation signals, means for receiving the electric reception signals, the processing and/or storage unit for the electric excitation and/or reception signals, means for the communication between the ultrasound system and a remote unit, and power supply means are included therein.

As an alternative it is possible to provide the probe to include both the array of transducers and the unit for processing and/or storing electric signals and to provide the electronic control unit to include means for generating the electric excitation signals, communication means and power supply means. Otherwise inside the probe there can be provided the array of transducers, the processing and/or storage unit, signal generating means and the control electronics can act only for powering the entire system and for communicating data processed by the probe to a remote unit, or as described above, for saving such data on a storage unit.

In particular as the system is a portable system, power supply means are preferably composed of a source generating and storing electrical power, of the battery type or the like, that can be rechargeable, with the recharging circuits integrated also within means for temporary fastening to the body.

As a further embodiment it is possible to provide the probe to directly communicate with a display remote unit without the aid of the electronic control unit: in this case the probe will include the array of transducers, means for generating electric signals, the processing and/or storage unit, power supply means and communication means, that will be connected to an interface allowing data detected by the probe to be transmitted to a remote display unit according to the methods described above.

As an alternative it is possible to provide communication means to transmit data to the remote display unit according to a wireless mode: apart from the case in which both communication means and the processing unit are introduced within the probe or within the control electronic unit, the fact of providing inside the processing unit a unit intended for reducing the bandwidth occupied by the transmitted signal is preferred such that the transmission between the portable ultrasound system and the remote display unit is facilitated. An example may be a unit generating video signals obtained from images deriving from the processing of received signals.

A particular arrangement for improving the attachment of the ultrasound system and for contemporaneously reducing the overall dimensions inside the monitoring system according to the present invention, provides the ultrasound probe to comprise an outer case having two side thinner and longer faces and two wider and longer faces, parallel each other and oriented with their length dimension in the direction of a longitudinal axis and provides the array of electroacoustic transducers to be arranged on one of said two wider faces such that the two faces are oriented substantially perpendicularly to the direction of propagation of the acoustic beam emitted from the probe. Such configuration allows the probe to be more thin and to be easily arranged inside the means for temporary fastening to the body by also using the housing pockets described above.

The outer case is in the form of a parallelepiped, which has six faces, each face has a rectangular form, and if each faces is constituted by a rectangle the two side thinner and longer faces and two wider and longer faces are univoquely identified, because there are only two faces that are both thinner and longer as there are only two faces that are wider and longer.

A preferred embodiment provides the portable ultrasound system to be used in combination with an electrocardiograph device, such that the monitoring system has two monitoring devices. Like the ultrasound system, the electrocardiograph is a portable device too, attached to the body part to be examined by means of the same means for temporary fastening to the body described above for the ultrasound system. The electrocardiograph is composed of a signal recording unit recording signals transmitted from one or more electrodes.

According to a first embodiment the electrodes are fitted on the recording unit, which is consequently integral therewith such that both the recording unit and the electrodes are placed inside the same housing pocket. As an alternative it is possible for the electrodes to be in communication with the recording unit without being fitted thereon, in this case several housing pockets will be provided.

Finally according to an improvement of the present invention it is possible to provide the recording unit of the electrocardiograph to be provided inside the electronic control unit of the portable ultrasound system, such to reduce the amount of devices of the monitoring system according to the present invention to a minimum. In this case inside the control electronic unit it is possible to provide processor means executing a logic processing program intended for establishing a connection among data received from the ultrasound system and the electrocardiograph.

Advantageously the means for temporary fastening to the body are composed of garment-like wearable elements such that a greater portability of the monitoring system of the present invention is guaranteed.

For example such fastening means can be composed of a band element intended for surrounding at least partially the body part to be monitored. Such element can be a closed element and in this case it is preferably composed of elastic material such that it can better adhere to the body such to guarantee the monitoring device to be integral with the body part to be examined and therefore not to be subjected to changes in its position due to movement during the normal walking motion, while being highly capable of fitting any part of the body to be examined.

A possible variant embodiment provides the band element to be an open element and to have two ends engaging each other by being, at least partially, overlapped along a closure that can be composed of standard closures of the clothing industry, such as for example snap fasteners, zip fasteners or hook-and-loop elements of Velcro type.

A further embodiment provides the fastening means to be composed of real garments, such as for example waistcoat, shirt, T-shirt, bra, such that constituent elements of the ultrasound system and/or of the electrocardiograph are arranged within such garments according to the methods described above. According to an improvement the fastening means are composed of garments at least partially composed of a band element, elastic or not, such that at least a part of the costituent elements of the ultrasound system and/or of the electrocardiograph such as for example the array of transducers and/or the electrodes are adherent to the body part under examination.

As described above, on the inside of the temporary fastening means, at the portion in contact with the body under examination, there are provided pockets housing the costituent elements of the ultrasound system and/or of the electrocardiograph. Such housing pockets can advantageously be composed of pockets cooperating with the inner part of the fastening means by means of a connecting interface that can be composed for example of detachable elements, such as for example the two parts of a snap fastener or two hook-and-loop elements of Velcro type. Therefore it is possible to provide all the inner surface of the temporary fastening means to be composed of Velcro type material, such that the housing pockets can be adjusted throughout all the surface such to fit the different morphologies of the patients and the different parts of the body to be examined.

A further embodiment provides such housing pockets to have through loops on the surface in contact with the fastening means which slidably engage a belt attached to the surface of the fastening means, such that such housing pockets can be adjusted throughout the surface.

One embodiment of the invention relates also to a general monitoring system comprising a device for monitoring parameters of a body or a part thereof under examination, an electronic control unit for signals from said monitoring device and means for supporting said device, which means are composed of means for temporary fastening to the body, which are composed in turn of garment-like wearable elements.

Even in this case the fastening means provide all embodiments and characteristics described above, and also those that can be deduced from those described above, such that as a consequence a monitoring system is provided allowing examinations to be performed by using portable systems such to provide the minimum bulkiness and the maximum comfort for the user, while maintaining unchanged the quality of data obtained from the performed examination, further having several possibilities for storing and displaying collected data.

A further improvement provides the use of two different devices for monitoring two different parameters and/or physiological characteristics, which devices are firmly integrated within the garment-like elements described above.

One embodiment of the present invention relates also to an ultrasound probe, intended particularly for diagnostic purposes, comprising an array of electroacoustic transducers, each one of which emitting ultrasound waves when powered with an electric excitation signal and generating an electric reception signal when impinged by an ultrasound wave or pulse generated for example by the reflection of ultrasound waves emitted therefrom, and an outer case having two side longer and thinner faces and two wider and longer faces, parallel each other and oriented with their length dimension in the direction of a longitudinal axis. The array of electroacoustic transducers is arranged on one of the two wider faces such that the two faces are oriented substantially perpendicularly to the direction of propagation of the acoustic beam emitted from the probe. This configuration allows the probe to be handled easier considerably decreasing its size. In addition the fact that it is possible to collect data by using one of the two wider faces and not a thin surface makes the probe highly capable of being adapted in the case of portable ultrasound systems, where the case of the probe has a wider surface in contact with and fastening the body without being affected by the movements of the patient when performing the examination.

According to a possible variant embodiment such probe is provided in combination with supporting means that are composed of means for temporary fastening to the body which are composed in turn of garment-like wearable elements. It has to be noted that even in this case the fastening means provide all embodiments and characteristics described above, and those that can be deduced from the previously described ones.

According to a particular embodiment such probe has the typical structure of the so called ultrasound probes and therefore it can be provided in combination with a remote display unit thus forming an ultrasound system comprising the array of transducers wherein each transducer is provided with at least a line for the communication with a processing unit through which the transducer feeds reception signals to a processing unit, and with a communication line through which electric excitation signals produced by a generating unit are fed to the transducer for excitating the transducer to emit ultrasound waves. The generating unit comprises means for generating excitation signals and means for feeding signals to the individual transducers according to one or more time sequence, so called transmitting beamformers, while the processing unit comprises means for combining the components of the reception signals of the individual transducers, which are received according to predetermined reception time window sequences, which time windows are different for each transducer, the so called receiving beamformers, for combining the components of the reception signals of the individual transducers deriving from a specific reflector having a specific position with reference to the transducers in the image detection plane. Moreover there are provided means for powering the probe and communication means for transmitting signals sent and received between the probe and the remote display unit. According to such variant embodiment inside the probe in addition to the array of transducers there are also provided communication means that can be of cable type or can transmit data to the remote unit in wireless mode, for example using the radio wave transmission.

In order to use the wireless communication and in order to make the probe as a portable device, according to a further improvement it is possible to provide inside the probe also means for generating electric signals and the unit for processing transmitted signals, as well as power supply means that in this case can be composed of a power generating and storing source of the battery type or the like.

Further arrangements can cause the inner structure of the probe to be modified for example by providing to use only one transmitting channel and only one receiving channel for the communication among the array of transducers and signal generating means and the processing unit. In this case it is necessary to provide switching means for alternately connecting each individual transmitting and receiving transducer to generating means and to the processing unit respectively, which unit controls also the operation of such switching means.

Moreover in order to improve the wireless transmission and reception it would be better to provide inside the probe, preferably inside the processing unit, means for converting reception signals into image data from which video signals are obtained, which occupy a lower transmission frequency band.

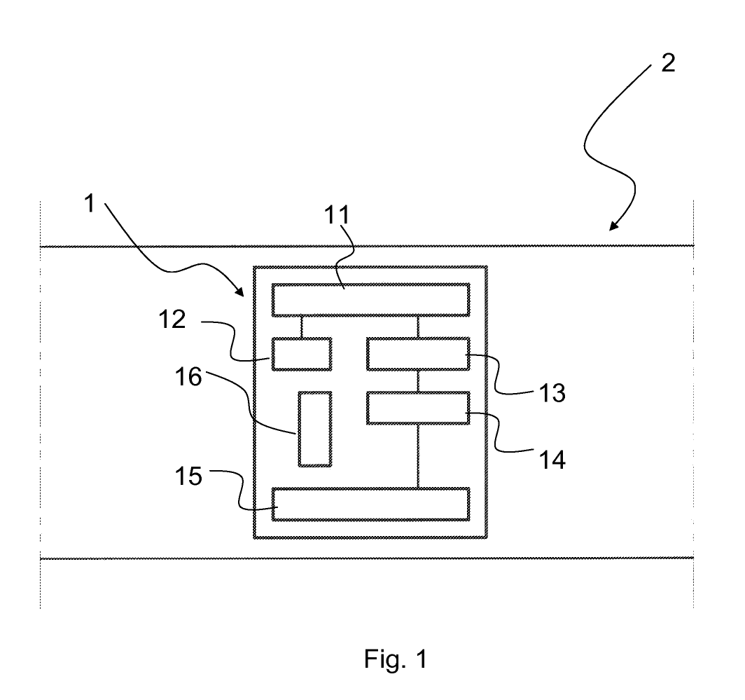

FIG. 1 schematically shows a possible configuration of the monitoring system according to the present invention comprising a portable ultrasound system 1 which is in turn provided with an array of electroacoustic transducers 11, each one of which emitting ultrasound waves when powered with an electric excitation signal and generating an electric reception signal when impinged by an ultrasound wave or pulse generated for example by the reflection of ultrasound waves emitted therefrom. There are also provided means for generating electric excitation signals 12, as well as means for receiving electric reception signals 13 and processing and storage units 14 for said electric excitation and reception signals.

Such processing means can comprise means for generating image data from reception signals and/or also means for generating images from image data, which images can be displayed on local or remote displays. Processing means can also comprise units for processing/generating excitation pulses for transducers in order to control them in emitting ultrasound pulses having specific characteristics such as for example the duration, the frequency spectrum, the waveform, relative phase displacements among different transducer elements and other characteristics that can be typically modified in ultrasound pulses emitted from ultrasound probes.

Moreover there are provided communication means 15 for the transmission and reception of the electric excitation and reception signals between the portable ultrasound system 1 and at least a remote unit for displaying, storing and further processing, as well as power supply means 16 for the whole portable ultrasound system 1.

In order to place the portable ultrasound system 1 at specific locations of the body to be examined it is provided in combination with supporting means composed of further means 2 for temporary fastening to the body such that at least the array of transducers 11 is fastened in the specific position for performing the examination and therefore it is integral with the body, or the part thereof, to be examined.

According to possible variant embodiments of the monitoring system according to the present invention, the means 2 for temporary fastening to the body can be provided not only for fastening the array of transducers 11 in a specific position, but for allowing one or more of the costituent elements of the portable ultrasound system 1 to be additionally positioned.

In particular according to the variant embodiment of FIG. 1 all the constituent elements are arranged throughout the surface of the fastening means 2 and each element communicates with another one by communication lines 17.

Thus means for generating electric excitation signals 12, means for receiving electric reception signals 13, the processing and/or storage unit 14 for the electric excitation and/or reception signals, communication means 15 and power supply means 16 as well as the array of transducers 11 are fastened by the fastening means 2.

Communication lines 17 are provided integrated within the fastening means 2 too and transmit data among the several elements of the ultrasound system 1 by using cables.

According to a variant embodiment the communication lines allow the several constituent elements to communicate in wireless mode, for example by using radio wave transmission.

It is also possible to provide an interface in combination with communications means 15 such that data detected by the portable ultrasound system 1 can be transmitted to a remote display unit Like communication lines 17, the transmission can be performed by using cables or according to a wireless mode, as a consequence such interface can be composed of a connector connected to a predetermined input of the remote unit, or can be composed of a radio wave antenna for wireless transmission. A further variant embodiment provides such interface to be composed of a storage device, which, in the perspective of reducing the overall dimensions, can be composed of a memory card of the Secure Digital type or the like, which records data detected by the monitoring system 1, stores them and displays them once it is inserted within the remote display unit.

In FIG. 1 the fastening means are schematically shown as a band to be wound and fastened around the body under examination with sensors and particularly with electroacoustic emitting and receiving transducers arranged at the organ to be examined that is at the anatomical window for performing the ultrasound scan of the organ to be examined. As it will be described below the band will have closure and tensioning means for example in the form of buckles and/or devices for removably interlocking the two ends of the band which can comprise also devices known under the brand name of Velcro®.

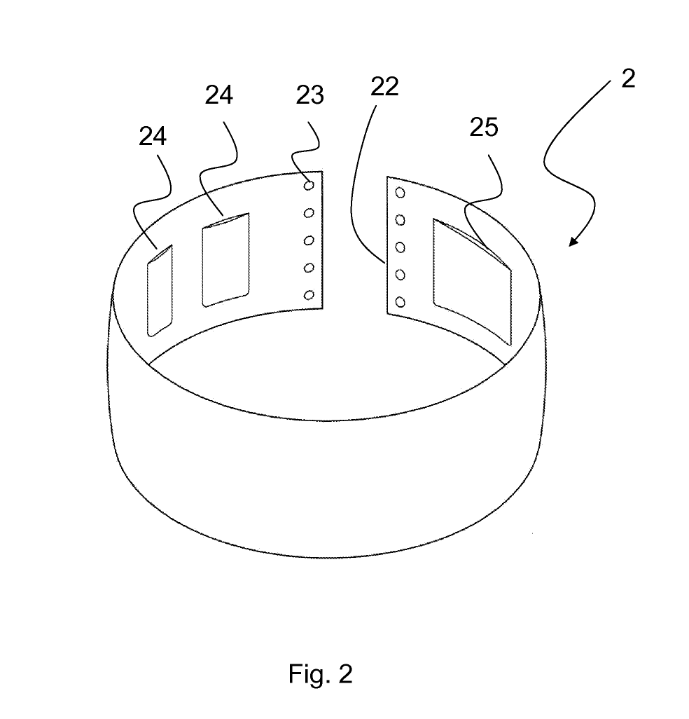

FIG. 2 shows a particular configuration of the fastening means 2, wherein such means are composed of garment-like wearable elements. With a particular reference to FIG. 2, the fastening means 2 are composed of a band element intended for surrounding at least partially the part of the body to be monitored. Such element is open and has two ends 21 and 22 engaging one another by being at least partially overlapped along a closure composed of snap fasteners 23.

Possible embodiments of the closure for such element can be standard closures used in the clothing industry, such as for example any buttons, zip fasteners or hook-and-loop elements of the Velcro® type.

Moreover the fastening means 2 of FIG. 2 on the inside thereof, at the part in contact with the body to be examined, have one or more housing pockets 24 intended for housing and keeping in place one or more of the costituent elements of the portable ultrasound system. Inside the fastening means 2 there are also provided elements 25 intended for keeping one or more of the costituent elements of the portable ultrasound system in a fixed position too: such elements are arranged throughout the inner surface, or a portion thereof, of the fastening means 2 and are composed of a first part that is integral with the band and cooperating with and engaging a second part, which is attached to the several elements of the portable ultrasound system. In particular in FIG. 2 such elements 25 are composed of hook-and-loop elements of velcro type, but a possible variant embodiment for example provides the use of the two parts of snap fasteners.

Likewise a variant embodiment of the fastening means 2 according to the present invention, provides the housing pockets 24 not to be fixed, but to be composed of pockets cooperating with the inner part of the fastening means 2 by a coupling interface that can be composed of removable temporary fastening means, such as for example the two parts of a snap fastener or two cooperating and hook-and-loop detachable fastening elements of the type known under the brand name of Velcro®.

Therefore it is possible to provide all the inner surface of the temporary fastening means 2 to be composed of Velcro type material, such that the housing pockets 24 can be adjusted along the surface such to fit different patient morphologies and different body parts to be examined.

As an alternative one or more strip-like regions can be provided wherein one of the removable fastening elements of the means for removably fastening the pocket or pockets throughout the garment is provided, while the other element is provided on the removable pockets.

The above characteristics allow the positioning especially of sensors and particularly of the arrays of emitting and receiving transducers to be optimized with respect to the body of the patient a wearable garment fitting not only the patient size, but also morphological and anatomical differences that usually occur from one patient to another patient and therefore signals obtained by the examination are optimized.

Obviously it is also possible to provide the garment to have a plurality of pockets firmly attached to the garment each one at a different location.

A further embodiment provides such housing pockets 24 on the surface in contact with the fastening means 2 to have through loops slidably engaging along a belt that is provided attached to the surface of the fastening means 2, such that such housing pockets 24 can be adjusted throughout the surface.

The band element forming the fastening means 2 is preferably composed of elastic material and likewise according to a further improvement it can be a closed, endless continuous element fitting the size of the different bodies, or part thereof, to be examined by means of the elastic properties of the used material allowing the several elements of the ultrasound system 1 to be integral with the part of the body to be examined and not to be subjected to changes in their positions due to movement during the normal walking motion, while being highly capable of fitting any part of the body to be examined.

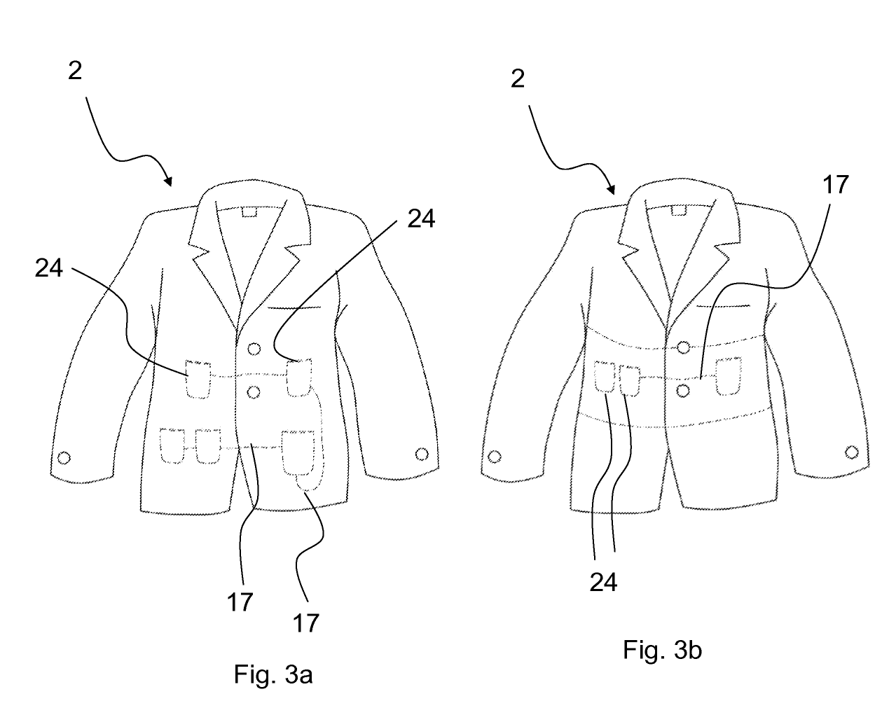

FIGS. 3a and 3b show two particular configurations of the monitoring system according to one embodiment of the present disclosure where the fastening means 2 are composed of garment-like elements. Particularly in FIGS. 3a and 3b the garment is a jacket, but further embodiments can provide the fastening means 2 to be waistcoat, shirt, T-shirt, bra or any garment.

In FIG. 3a the costituent elements of the portable ultrasound system described in FIG. 1, are arranged throughout the inner surface of the whole garment, communicating each other by means of communication lines 17 and are kept in a fixed position by means of housing pockets 24.

As described above such housing pockets 24 can be provided in a fixed position or can be movable throughout all or a part of the inner surface of the jacket. In this last case such housing pockets 24 are composed of pockets cooperating with the inner part of the garment by means of a coupling interface that can be composed of detachable elements, such as for example the two parts of a snap fastener or two hook-and-loop elements of Velcro type.

As an alternative such housing pockets 24 on the surface in contact with the inner surface of the jacket have through loops slidably engaging along a belt that is provided attached to the inside of the jacket, such that such housing pockets 24 can be adjusted throughout the surface.

FIG. 3b instead shows a jacket partially composed of a band forming the fastening elements 2 and having all the characteristics previously described with reference to FIG. 2.

It is also possible to provide an interface connected to one or more of the constituent elements of the ultrasound system 1, such that data detected thereby can be transmitted to a remote display unit. Like communication lines 17, the transmission can be performed by using cables or according to a wireless mode, as a consequence such interface can be composed of a connector connected to a specific input of the remote unit, or can be composed of a radio wave antenna for wireless transmission.

A further variant embodiment provides such interface to be composed of a storage device, which, in the perspective of reducing the overall dimensions, can be composed of a memory card of the Secure Digital type or the like, which records data detected by the monitoring system 1, stores them and displays them once it is inserted within the remote display unit.

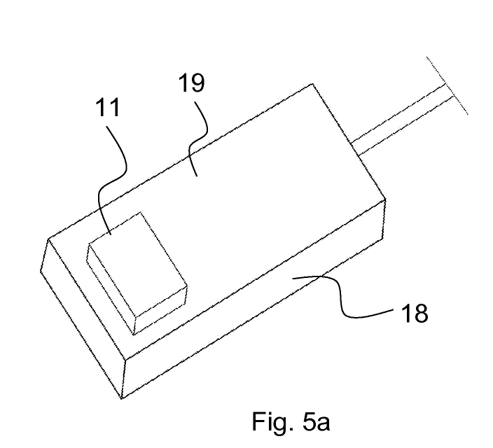

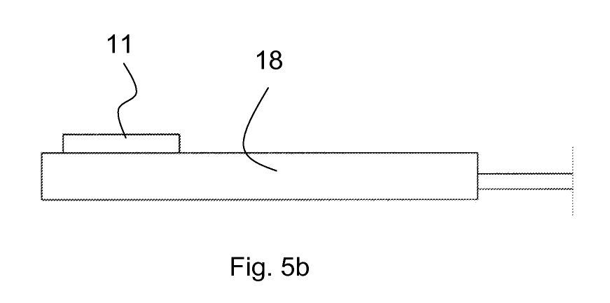

FIGS. 5a and 5b show a particular configuration of the portable ultrasound system 1 of the monitoring system of the present invention where the ultrasound system is composed of an ultrasound probe and of control electronics.

The ultrasound probe has an outer case having two side thinner and longer faces 18, and two wider and longer faces 19, parallel each other and oriented with their length dimension in the direction of a longitudinal axis. Moreover the array of transducers 11 is provided fitted on one of the two wider faces 19 such that the two faces 19 are oriented substantially perpendicularly to the direction of propagation of the acoustic beam emitted from the probe.

According to a first embodiment, both the ultrasound probe and the control electronics are housed within the housing pockets of the fastening means and there are several possible configurations providing the constituent elements of the portable ultrasound system 1 inside the ultrasound probe and/or the control electronics, and these are all the subject of the present invention.

In particular only the array of transducers 11 can be provided within the probe, that by means of communication lines 17 receives and sends signals from and to the control electronics and consequently means for generating electric excitation signals 12, means for receiving electric reception signals 13, the processing and/or storage unit 14 for the electric excitation and/or reception signals, communication means 15, and power supply means 16 are included therein.

As an alternative it is possible to provide the probe to include both the array of transducers 11 and the processing and/or storage unit 14 for the electric signals and to keep inside the electronic control unit means for generating the electric excitation signals 12, means for receiving the electric reception signals 13, communication means 15 and power supply means 16. Otherwise the array of transducers 11, the processing and/or storage unit 14, signal generating means 12 can be provided inside the probe and the function of the control electronics can be only to power the whole system and to communicate the data processed by the probe to a remote unit, or as previously described, to save such data on a storage unit.

In particular the power supply means 16 can be composed of a source generating and storing electrical power, of the battery type or the like, that can be rechargeable, with the recharging circuits integrated within the means for temporary fastening to the body too.

As a further embodiment it is possible to provide the probe to directly communicate with a remote display unit without the aid of the electronic control unit: in this case the probe will include the array of transducers 11, means for generating electric signals 12, means for receiving the electric reception signals 13, the processing and/or storage unit 14, power supply means 16 and communication means 15, that will be connected to an interface allowing data detected by the probe to be transmitted to a remote display unit according to the methods described above.

As an alternative it is possible to provide communication means 15 to transmit data to the remote display unit according to a wireless mode: apart from the case in which both communication means 15 and the processing unit 14 are inserted within the probe or the control electronic unit, the fact of providing inside the processing unit a unit intended for reducing the bandwidth occupied by the transmitted signal is preferred such that the transmission between the portable ultrasound system 1 and the remote display unit is facilitated. An example may be a unit generating video signals obtained from images deriving from the processing of received signals.

An example of the technology concerning a wireless probe that has to be considered as not limitating the several possible configurations of the system is described in EP 2164398 and WO2008/066681, both of which are incorporated by reference.

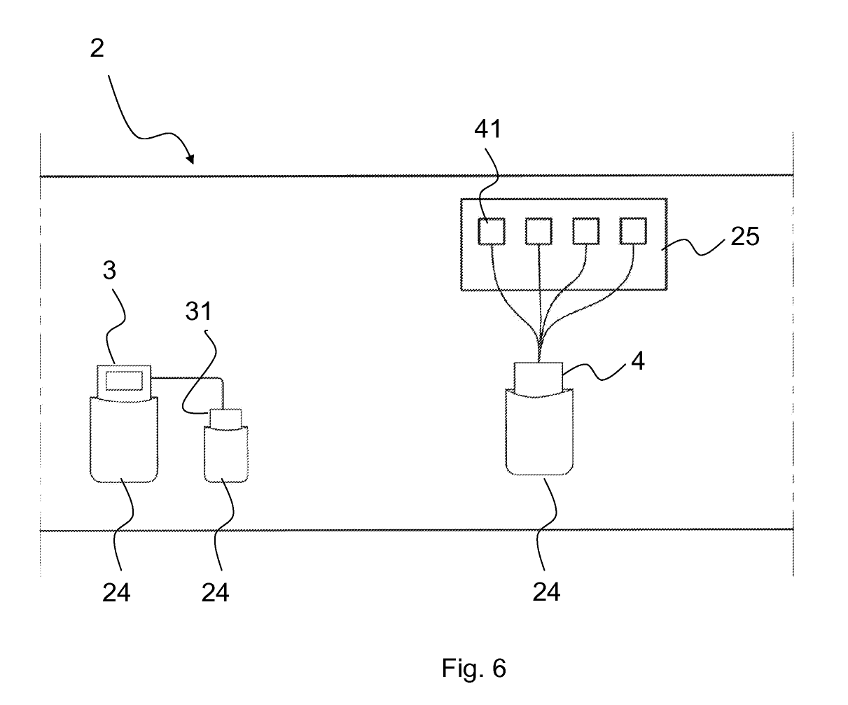

FIG. 6 schematically shows a possible variant embodiment of the monitoring system of the present disclosure where the portable ultrasound system 1 is provided in combination with an electrocardiograph 3. The monitoring device 1 is composed of an ultrasound probe 3 and control electronics 31, according to characteristics widely described above in FIG. 5, and it is provided in combination with an electrocardiograph device. Such electrocardiograph device is composed of a signal recording unit 4 recording signals transmitted from one or more electrodes 41.

With a particular reference to FIGS. 5a and 5b, both the portable ultrasound system and the electrocardiograph device are supported by means 2 for temporary fastening to the body, which have all the characteristics concerning the fastening means previously described in FIG. 2.

In particular the ultrasound probe 3, the electronic control unit 31 and the recording unit 4 are housed within the housing pockets 24, while sensors 41 are supported by elements 25 which are composed of hook-and-loop elements of Velcro type cooperating one with the other a first part of which being integrally attached to each electrode 41, while a second part is provided on the inner surface of the fastening means 2.

Even in this case both the housing pockets 24 and the elements 25 have all the characteristics described in FIG. 2.

According to a further embodiment the electrodes 41 are fitted on the recording unit 4, which is consequently integral therewith such that both the recording unit 4 and the electrodes 41 are placed within the same housing pocket 24 or are fitted on the same element 25.

Finally according to an improvement of the present invention it is possible to provide the recording unit 4 of the electrocardiograph to be provided within the electronic control unit 31 of the ultrasound probe 3. In this case it is possible to provide processor means inside the electronic control unit for executing a logic processing program intended for setting a connection among data received from the ultrasound probe 3 and from the electrocardiograph.

Even in this case the monitoring system according to the present invention that, with a particular reference to FIG. 6, provides the use of two different monitoring devices, the ultrasound probe and of the electrocardiograph, can use an interface for transmitting data detected by the two different monitoring devices to a remote display unit. The transmission can occur by using cables or in wireless mode, consequently such interface can be composed of a connector connected to a specific input of the remote unit, or can be composed of a radio wave antenna for the wireless transmission. A further variant embodiment provides such interface to be composed of a storage device, which, in the perspective of reducing the overall dimensions, can be composed of a memory card of the Secure Digital type or the like, which records the received data, stores them and displays them once it is inserted within the remote display unit.

On the remote display unit will be possible to display in a diversified manner the received signals, or it will be possible to set a functional connection among the received signals by means of the processing program provided inside the control unit in common to the two monitoring devices.

Finally, the two monitoring devices can also advantageously display the received and recorded signals independently from the remote display unit, by means of a screen fitted on the control electronics 31 and on the recording unit 4 respectively.

With reference to the several shown embodiments it is possible to provide the several operating units of the diagnostic systems, such as the ultrasound system, the ECG system or the ultrasound and ECG combined system to be divided in several separated physical units and to be arranged at different locations with reference to the extension and the shape of the fastening means, a dedicated housing pocket being provided for each physical unit having dimensional and electrical properties adapted for the needs of the units they have to house. Obviously the cables connecting said physical units have to be integrated within or associated to the fastening means.

To this aim said cables can be provided housed into suitable channels called coulisse provided within the fastening means and within which the cables can be threaded and unthreaded or firmly inserted. The latter at their ends in turn will have terminals connecting to corresponding complementary connectors provided on the physical units.

A particular embodiment can provide a plurality of parallel coulisses housing the connection cables along the extension of the fastening means and for example parallely to the extension of the removably fastening means of the pockets 24, which coulisses have a wall opposite to the means 2 for fastening to the patient body which is not continuous, that is it is provided with a plurality of inlet and outlet apertures for the cable housed within the coulisse. Said row of apertures allows the cable to enter and come out from the coulisse at different locations thereof and therefore the length of the cable housed within the coulisse to be adjusted to the variable distances among the physical units since said units can be placed at a plurality of different positions with reference to the shape and the surface extension of the means for fastening the system to the body under exmination.

When pockets are fixed, then the coulisses can be such that the pockets are directly connected one another.

As an alternative, it is possible to provide cables whose outer covering jacket has an outer surface covered or provided at least at partial areas with removable fastening means of the Velcro® type or the like which are intended for cooperating with the complementary removable interlocking means provided on the wearable fastening means.

Likewise a possible variant embodiment of the monitoring system of the present invention provides the portable ultrasound system to be provided in combination with elements for temporary fastening to the body composed of a band element comprising rigid elements in combination with flexible elements.

Rigid elements can act as the housing pockets 24 and can allow one or more of the elements of the portable ultrasound system to be housed, while coulisse-like seats are housed within the flexible elements for receiving the communication lines 17 for the communication among the several constituents.

Likewise the several described embodiments, both the portable ultrasound system and the means for temporary fastening to the body have all the characteristics already previously widely described, in particular whether the ultrasound system is a wireless ultrasound probe, it is advantageous for the rigid elements to have slots therein. Such slots allow the battery to be inserted within the probe without the need of taking the probe out from the housing, or they can be apertures for connecting the whole system to interfaces intended for example for the connection to an external mains supply for recharging the battery, or for further devices storing the data detected by the probe.

Moreover flexible elements can not only house the communication lines, but they can be the communication contacts among the several components of the portable ultrasound system: such configuration allows a modular structure of the fastening elements to be obtained comprising a series of rigid elements housing the components of the portable ultrasound system which are alternated with the flexible elements.

Finally preferably, but not exclusively, the band element, particularly the rigid elements, is composed of an elastically deformable material, for example rubber materials, allowing the components housed therein to be protected while remaining highly capable of fitting the body part and/or the patient to be examined.

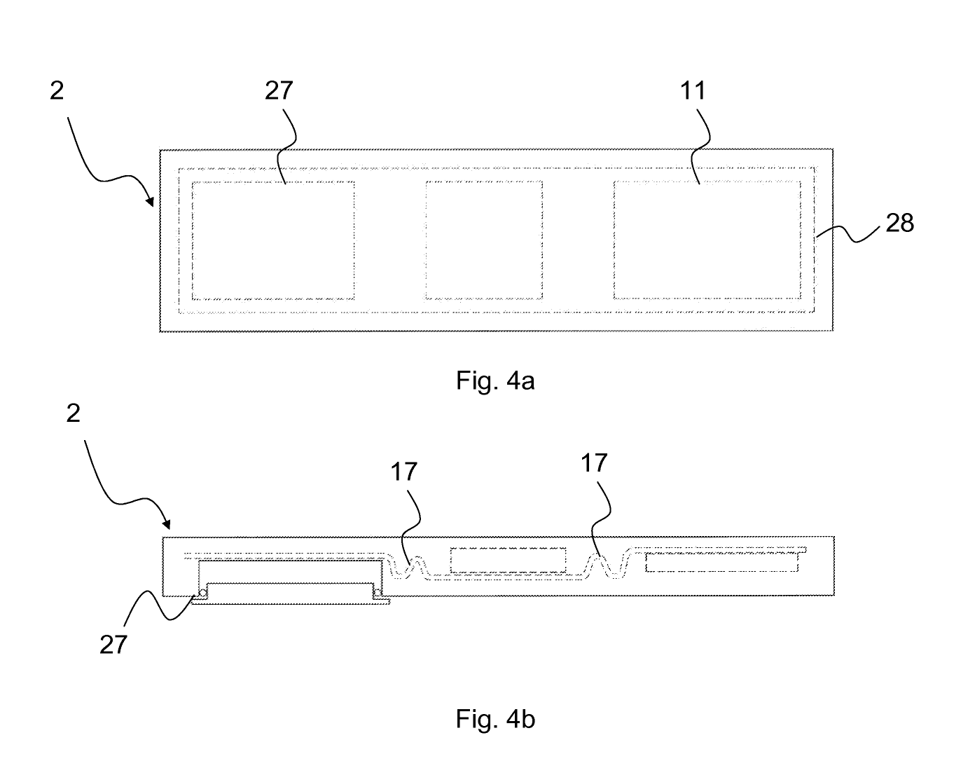

In particular FIGS. 4a and 4b show a possible variant embodiment of the monitoring system of the present invention. According to such configuration the several components of the portable ultrasound system are provided in combination with means 2 for temporary fastening to the body composed of a band element: such components are fitted embedded within the material of the band element 2.

Thus the band element 2, in addition to act for the temporary fastening to the body acts also as a casing for components of the portable ultrasound system thus replacing the real casing containing them. The means for temporary fastening to the body in the inside part in contact with the body can have a slot allowing the array of transducers 11 to contact the body part to be examined. The array of transducers 11 is connected to the remaining components, already described above, of the ultrasound system which are integrated within the band element and connected each other by means of the communication lines 17 also integrated within the same element. Advantageously the band element provides a containing enclosure element 27 that can be opened for housing the battery, opening on the top in the direction opposite to the inner part in contact with the body under examination, for taking out and introducing the battery and/or for connecting the interfaces already described above.

According to an improvement of the variant embodiment shown in FIGS. 4a and 4b the material of the band element 2 is a deformable and waterproof material, for example a rubber material, while the containing enclosure element 27, for housing the battery has a watertight closure such to insulate the whole system against possible infiltrations.

The slot is likewise provided on the inner surface of the fastening means in contact with the body and it surrounds the array of transducers 11 at the side perpendicular to the end side thereof, which is in contact with the body part to be examined too, such that the end side is flush with the rubber material of the fastening means 2 surrounding the array of transducers. Moreover the slot is made in a watertight manner such that the interface part between the rubber part and the array of transducers 11 does not allow liquids to penetrate into the means 2 for temporary fastening to the body.

With a particular reference to FIGS. 4a and 4b, the array of ultrasound transducers 11 is also embedded inside the band element 2, without providing any slots such that the rubber element of the band element 2 can be passed by ultrasounds and it acts both as the protection and as the coupling means between the array of transducers 11 and the body part under examination.

According to an improvement shown in FIGS. 4a and 4b, an inner receiving unit 28 is provided, embedded within the fastening means 2 too, for the communication between the portable ultrasound system and one or more of the remote control devices intended for example for setting operation parameters of the portable ultrasound system. As an alternative to said inner receiving unit 28, it is possible to provide a push-button panel made within the fastening means 2 composed of pressure elastic areas made as one piece with the rubber material of the band 2.

Similarly to what said above even the portable ultrasound system and the means for temporary fastening to the body described in FIGS. 4a and 4b have all the characteristics of the several previously described embodiments of the monitoring system of the present invention.

Again according to a possible variant, with reference to the embodiment of FIGS. 5a and 5b, it is possible to provide the ultrasound probe to be made of two parts one of which composed of the array of tranducers, fitted on a bearing element provided with a multipolar terminal for the connection to a complementary connection terminal provided on the remaining part of the probe which is intended for housing the electronic units the two connectors establishing a communication between the transducers of the array of transducers and said electronic units. By means of such arrangement it is possible to replace the array of transducers adapting it to the examination requirements. In particular therefore on the same probe using common electronic units it is possible to fit linear or two-dimensional arrays of transducers and having a different number of elements or dimensional extensions.

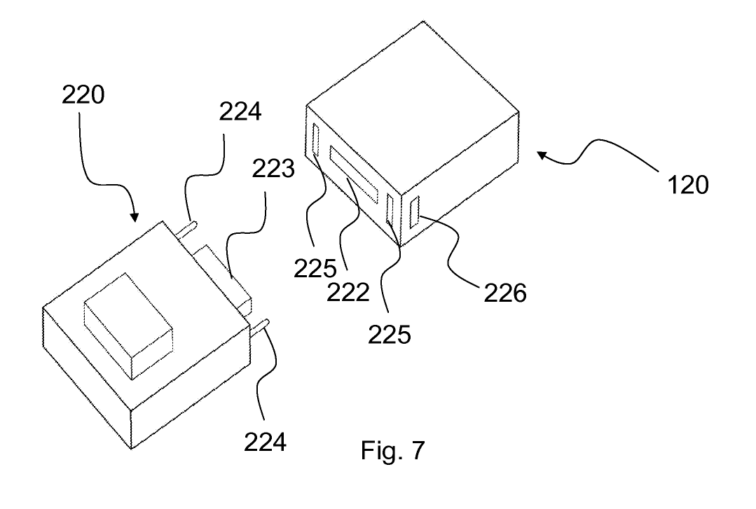

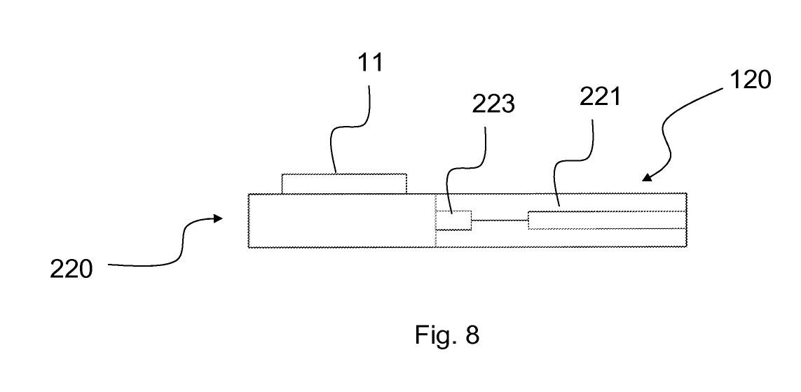

Another embodiment is illustrated in FIGS. 7 and 8. Such example has not to be considered as a limitation. Several configurations are possible and fall within the range of the person skilled in the art as an obvious choice among different options.

In particular the array of transducers that is shown can be replaced without the need of replacing also a part of the probe case. However if the replacement is performed by people not skilled in the probe construction the shown solution will be more convenient and safe.

In the example of FIGS. 7 and 8, a probe of the type described in FIGS. 5a and 5b has a case 20 divided into two parts 120 and 220 that can be mechanically removably coupled together for example by means of removable interlocking means provided on the two parts 220 and 120 of the case 20. Said means can be of any type and can be selected among the several variants known to and available to the person skilled in the art.

In FIGS. 7 and 8 said means are schematically shown by male elements 224 having an extension in the direction for mutually connecting/releasing the two case parts 120, 220 having a tooth end on one case part 220 intended for being engaged behind a mating element provided within an interlocking seat 225 in the other case part 120. Button means 226 can be controlled from the outside in order to bend the male elements 224 in a position releasing the tooth end thereof and so allowing them to be taken out from the seats 225 and allowing the two parts of the case 220 and 120 to be moved away one with respect to the other.

A case part 220 bears the array of transducers 11. Each transducer element is connected by a line with a pole of a connection terminal 223 (in this case a male) provided on the case part 220 at the face intended to adhere against the corresponding face of the case part 120 with the two case parts 120, 220 in a coupled condition.

The connection terminal 223 has an electrical pole for each communication line of each transducer element of the array 11 and it cooperates with a complementary connection terminal 222 provided on the face of the case part 120 intended to contact the face of the other case part 220.

The position of the two connection terminals 222, 223 and even the direction for being mutually engaged and released is advantageously the same necessary for mechanically coupling and releasing said two case parts 120, 220. By this arrangement by joining together the two case parts 120 and 220 the electrical contact between the two connection terminals 222, 223 and therefore between the individual transducer elements of the array of transducers 11 and the processing electronics 221 is automatically established. Such electronics can be configured according to one or more of the several variants described above or known from the prior art with reference to the greater or lower integration of operating units into the ultrasound probe.

As it is clear the construction allows the array of transducers to be replaced with other array of transducers that can be different as regards the number of transducers and the arrangement thereof inside the array. Considering the fact of making the electronics 221 and the connectors 222, 223 all equal each other and correspondingly to the array of transducers 11 providing as many allowable transducer elements as possible, the probe will be adapted in a very simple way to the provision of a different number of transducers in the array of transducers, by simply causing the contacts or poles of the connector 223 of the case part 220 bearing the array of transducers 11 to be not operating, therefore the channels of the electronic unit 221 corresponding to those of the non operating contacts of the connector 223 are not operating too.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected. It is also contemplated that structures and features embodied in the present examples can be altered, rearranged, substituted, deleted, duplicated, combined, or added to each other. The articles “the”, “a” and “an” are not necessarily limited to mean only one, but rather are inclusive and open ended so as to include, optionally, multiple such elements.

What is claimed is:

1. A portable ultrasound monitoring system comprising:
an array of electroacoustic transducers, each transducer is constructed and arranged to emit an ultrasound wave when powered with an electric excitation signal and generate an electric reception signal when impinged by a reflected ultrasound wave;
generating means for generating said electric excitation signals;
receiving means for receiving said electric reception signals;
processing and storage unit constructed and arranged to process and store said electric excitation signals and said electric reception signals;
communication means for delivering said electric excitation signals and said electric reception signals between said portable ultrasound system and a remote unit constructed and arranged to display electric signals;
power supply means; and
supporting means for supporting said portable ultrasound monitoring system, said supporting means having fastening means for temporarily fastening at least said array of electroacoustic transducers to a body under examination in a predetermined position for performing an examination.

2. The portable ultrasound monitoring system of claim 1, wherein said fastening means also fastens said generating means and said receiving means to the body under examination, at least one communication line is provided between said generating means and said receiving means and said array of electroacoustic transducers.

3. The portable ultrasound monitoring system of claim 2 further comprising an ultrasound probe and of an electronic control unit, said ultrasound probe housing said array of electroacoustic transducers.

4. The portable ultrasound monitoring system of claim 3, wherein a portion of said fastening means in contact with the body under examination has a housing pocket constructed and arranged to maintain said ultrasound probe and/or said electronic control unit in a fixed position.

5. The portable ultrasound monitoring system of claim 4, wherein said fastening means comprises a plurality of housing pockets, said housing pockets are provided at predetermined locations within said fastening means such that said generating means and said receiving means and said communication lines are integrated.

6. The portable ultrasound monitoring system of claim 1, wherein said fastening means comprises an interface for providing said communication means to said remote unit.

7. The portable ultrasound monitoring system of claim 3, wherein said ultrasound probe comprises an outer case having two opposed first faces having a first width and two opposed second faces having a second width wider than said first width, said first faces and said second faces are oriented with their length dimension in the direction of a longitudinal axis of the outer case, said array of ultrasound transducers are located on one of said second faces such that said second faces are oriented substantially perpendicularly to the direction of propagation of an acoustic beam emitted from the ultrasound probe.

8. The portable ultrasound monitoring system of claim 3, further comprising an electrocardiograph device.

9. The portable ultrasound monitoring system of claim 8, wherein said electrocardiograph device is composed of a recording unit upon which an electrode is fitted.

10. The portable ultrasound monitoring system of claim 8, wherein said electrocardiograph device is composed of a recording unit in communication with an electrode.

11. The portable ultrasound monitoring system of claim 10, wherein said recording unit is provided inside said electronic control unit.

12. The portable ultrasound monitoring system of claim 1, wherein said fastening means are composed of garment-like wearable elements.

13. The portable ultrasound monitoring system of claim 1, wherein said fastening means are composed of a band element constructed and arranged to at least partially surround a part of the body to be monitored.

14. The portable ultrasound monitoring system of claim 13, wherein said band element is made of elastic material such that said element can fit the different anatomical parts of the body under examination.

15. The portable ultrasound monitoring system of claim 1, wherein said fastening means are composed of an element comprising a band element.

16. The portable ultrasound monitoring system of claim 1, wherein a portion of said fastening means in contact with the body under examination has a plurality of Velcro-type hook-and-loop elements constructed and arranged to maintain said portable ultrasound system in a fixed position

17. A monitoring system comprising:
a first device for monitoring parameters of a body under examination;
an electronic control unit constructed and arranged to receive signals from said monitoring device;
and supporting means for supporting said device, said supporting means having fastening means for temporarily fastening device to a body under examination in a predetermined position for performing an examination, said fastening means are composed of garment-like wearable elements.

18. The monitoring system of claim 17, further comprising a second device, said first device and said second device are constructed and arranged to monitor different parameters of the body under examination, wherein said first device and said second device are firmly integrated within said garment-like wearable elements.

19. The monitoring system of claim 17, wherein said garment-like wearable elements comprise a plurality of housing pockets, said pockets are arranged at predetermined locations such that said first device is integrated.

20. The monitoring system of claim 17, wherein said fastening means comprises an interface for providing said communication means to a remote unit constructed and arranged to display electric signals.

21. An ultrasound probe for diagnostic purposes, said probe comprises:
an array of electroacoustic transducers, each transducer is constructed and arranged to emit an ultrasound wave when powered with an electric excitation signal and generate an electric reception signal when impinged by a reflected ultrasound wave; and
an outer case having two opposed first faces having a first width and two opposed second faces having a second width wider than said frist wide, said first faces and said second faces oriented with their length dimension in the direction of a longitudinal axis of the outer case,
wherein said array of ultrasound transducers are located on one of said second faces such that said second faces are oriented substantially perpendicularly to the direction of propagation of an acoustic beam emitted from the probe.

22. The ultrasound probe of claim 21 further comprising a supporting means for supporting said ultrasound probe, said supporting means having a fastening means for temporarily fastening said ultrasound probe, said fastening means are composed of garment-like wearable elements.

23. The ultrasound probe of claim 22 further comprising a communication means for delivering said electric excitation signals and said electric reception signals between said ultrasound probe and a remote unit constructed and arranged to display electric signals

24. The ultrasound probe of claim 23, wherein said communication means is of the cable type.

25. The ultrasound probe of claim 24, wherein said communication means is of the wireless type.

26. The ultrasound probe of claim 21 further comprising:
generating means for generating said electric excitation signals;
receiving means for receiving said electric reception signals;
processing and storage unit constructed and arranged to process and store said electric excitation signals and said electric reception signals; and
power supply means.
